(19) United States
(12) Patent Application Publication
(10) Pub. No.: US 2026/0041382 A1
(43) Pub. Date: Feb. 12, 2026

(54) METHOD FOR ASCERTAINING AN ITEM OF ENVIRONMENT INFORMATION BASED ON AN X-RAY IMAGE, PROCESSING FACILITY, ENDOSCOPY FACILITY, COMPUTER PROGRAM, AND DATA CARRIER

(71) Applicant: Siemens Healthineers AG, Forchheim (DE)

(72) Inventors: Alois Regensburger, Poxdorf (DE); Matteo Pantano, München (DE); Philipp Roser, Allston, MA (US)

(21) Appl. No.: 19/295,109

(22) Filed: Aug. 8, 2025

(30) Foreign Application Priority Data
Aug. 8, 2024 (DE) 10 2024 207 553.1

(51) Int. Cl. A61B 6/12; A61B 6/00; A61B 6/50; G06T 7/73; G06T 17/00

(52) U.S. Cl. CPC A61B 6/12; A61B 6/504; A61B 6/5211; G06T 7/73; G06T 17/00; G06T 2207/10068; G06T 2207/10121; G06T 2207/20081; G06T 2207/30004; G06T 2207/30052; G06T 2210/41

(57) ABSTRACT

A computer-implemented method for ascertaining an item of environment information is provided. The item of environment information relates to material in surroundings of a third article and/or an interaction of the third article with the material. The method includes receiving an X-ray image and an item of sensor information, and determining model parameters or limiting possible parameter values of the model parameters of a three-dimensional model of the third article as a function of the X-ray image in order to specify an X-ray-dependent model. The three-dimensional model describes a three-dimensional shape and/or pose of the third article as a function of the model parameters. The method includes ascertaining the item of environment information as a function of the X-ray-dependent model, where the item of environment information and/or the X-ray-dependent model additionally depends on the item of sensor information. The item of environment information is provided.

This application claims the benefit of German Patent Application No. DE 10 2024 207 553.1, filed on Aug. 8, 2024, which is hereby incorporated by reference in its entirety.

### BACKGROUND

The present embodiments relate to ascertaining an item of environment information.

In the framework of a medical treatment, it may frequently be relevant to obtain items of information about the environment of a third article located inside a patient (e.g., a medical implant or instrument). Thus, for example, it may be highly relevant to identify the presence of particular biological material (e.g., a calcification) or a blood clot in an implant, or the presence of an object that is to be removed (e.g., a gallstone) in an endoscopy basket. When positioning or checking a position of an implant, it may be relevant that biological material is located in the environment (e.g., in order to check whether there is sufficient contact with a vessel wall).

From the field of treating endovascular aneurysms, it is known to take into consideration the interaction of catheters with the surrounding vessel system in order to estimate a shape of a catheter that has been inserted. Such an approach is discussed, for example, in the publication Jäckle S. et. al, Instrument localization for endovascular aneurysm repair: comparison of two methods based on tracking systems or using imaging, Int J Med Robot. 2021; 17 (6): e2327. https://doi.org/10.1002/rcs.2327. However, since in this case, the assessment of the shape assumes that the catheter adapts to the shape of vessels, which shape is ascertained based on a preoperative CT scan, interactions of the catheter with the vessel system that actually occur cannot be identified or taken into consideration in this case. Accordingly, the identified shape does not make it possible to infer properties of the biological material surrounding the catheter.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, extraction of items of information about surroundings of a third article located inside a patient, such as an implant or a medical instrument, is enabled or improved.

In one embodiment, a computer-implemented method for ascertaining an item of environment information based on at least one two-dimensional X-ray image that maps at least one portion of a third article located inside a patient, and at least one item of sensor information that is based on at least one measured value of at least one sensor of the third article is provided. The item of environment information relates to material in the surroundings of the third article and/or an interaction of the third article with this material. The method includes the following steps: receiving the X-ray image and the item of sensor information; determining model parameters or limiting the possible parameter values of the model parameters of a three-dimensional model of the third article as a function of the X-ray image in order to specify an X-ray-dependent model, where the three-dimensional model describes a three-dimensional shape and/or pose of the third article as a function of the model parameters; ascertaining the item of environment information as a function of the X-ray-dependent model, where first, the item of environment information and/or second, the X-ray-dependent model additionally depend on the item of sensor information; and providing the item of environment information.

It has been found that a joint use of sensor data of a third article-side sensor and of the three-dimensional model parameterized as a function of the X-ray image makes it possible to accurately ascertain properties of material in the surroundings of the third article, or its interaction with the third article, even without capturing three-dimensional image data. For example, the item of environment information may relate to a biological or endogenous material or the interaction of the third article with biological or endogenous material. However, as an alternative or in addition, the item of environment information may also relate to synthetic materials (e.g., bone cement or embolization material used in the framework of an embolism in order to fill an aneurysm, such as embolism coils or an embolization material that hardens on contact with blood).

It is essential that as a first information channel, the X-ray image and as a second information channel, the item of sensor information are taken into consideration since considerable uncertainties may remain when only one of these inflation channels is used. Thus, while with a known embodiment of the third article, based on its three-dimensional shape and the forces acting on the third article that may be identified based on the shape, inferences may be made about the consistency or elasticity of the surrounding biological material, an assessment of the three-dimensional shape of the third article solely based on a single two-dimensional X-ray image, or also based on a series of a plurality of chronologically recorded, two-dimensional X-ray images with identical recording geometry, as are captured, for example, in the framework of fluoroscopy, may be too inaccurate for this. This may, for example, be ambiguous, so when the item of sensor information is not taken into consideration, typically at best, a rough and error-prone assessment of the item of environment information may be possible.

The item of sensor information also frequently does not in itself allow accurate inferences about local environment properties, since, for example, owing to the elastic deformability of the third article that may be given with medical third articles, the position of the sensor and partly also the relative position of various sensor components to each other, without use of the X-ray-dependent model, is not known or at best is very imprecisely known.

As will be explained in more detail later, the item of sensor information may, for example, supply additional items of information with respect to an instantaneous shape of the third article and/or in relation to forces that act on the third article owing to the surrounding material. An underdetermination or inaccuracy of the model parameters may be compensated hereby in that a consistency of the X-ray-dependent model is requested with the item of sensor information, so the X-ray-dependent model additionally depends on the item of sensor information. However, as an alternative or in addition, the item of sensor information may also be used directly when ascertaining the item of environment information, for example, in order to distinguish between different materials in the surroundings of the third article based on a measured force, impedance, conductivity, or the like.

All model parameters of the three-dimensional model may be determined from the X-ray image and optionally the item of sensor information. However, it is also possible that only a limitation of the possible parameter values of the model parameters may be achieved. For example, a system of equations may describe a correlation between items of image information that are ascertained based on the X-ray image and describe, for example, positions of a plurality of specified features in the X-ray image, and describe the model parameters, with the system of equations being underdetermined. In this case, the model parameters may be specified, for example, as a function of one or more free parameters, with the number of free parameters being reduced with respect to the number of model parameters. If, first, in order to limit the possible parameter values of the model parameters, solely the X-ray image is taken into consideration, the values of the free parameters, or at least limited value ranges for the free parameters, may thus be determined (e.g., based on the item of sensor information).

The model parameters and/or the free parameters may have discrete possible values. For example, in the case of an underdetermination (e.g., when prior knowledge is taken into consideration), only a number of parameterization options remain for the three-dimensional model or for at least a subgroup of its model parameters that are consistent with the X-ray recording. In this case, a discrete value of a free parameter may be allocated, for example, to each of the parameterization options.

The three-dimensional model may describe, for example, a boundary surface, described, for example, by triangles, which follows the contour of the third article. The coordinates of the corners of the triangles or other partial surfaces of the boundary surface may thus describe the boundary surface in the form of a coordinate grid.

As has already been mentioned above, a plurality of X-ray images that may be captured (e.g., with identical recording geometry) may also be taken into consideration for determining the model parameters or for limiting the possible parameter values of the model parameters. This may be expedient, for example, in order to utilize a temporal coherence of the movement of the third article via an observation at a plurality of instants. For example, ambiguities in the X-ray-dependent model that would remain if only one X-ray image was taken into consideration may be at least partially removed hereby.

The third article may, for example, be deformable, where at least one of the model parameters describes the deformation. For example, the model parameters, or at least one of the model parameters, may describe an elastic deformation of the third article. Based on the deformation (e.g., elastic deformation), it is possible to identify, for example, forces with which the surrounding material acts on the third article.

For example, based on such a deformation, it is possible to identify whether or which portions of the third article are deformed by walls of a cavity of the patient that receives the third article (e.g., a bile duct or a blood vessel), or whether solid organic material (e.g., a gallstone to be removed or a blood clot) is received in a cavity of the third article. Appropriate sensors that may measure, for example, deformations or forces may remove, for example, ambiguities when the model parameters are determined based on the X-ray image.

The identification of a deformation of the third article may also be essential to be able to estimate the exact position of the sensor and, for example, also a relative position of different sensor components of the sensor to each other. For example, sensors for measuring the conductivity of surrounding material may have electrodes that are spaced apart from one another as sensor components. Sensors that measure a relative electrical potential different positions to each other may also have a plurality of electrodes that are spaced apart from one another as sensor components.

Based on the X-ray-dependent model that describes the deformation, the significance of items of sensor information may thus be significantly increased (e.g., with respect to a resistance of the material and/or with respect to an inductance that is influenced by the material).

As an item of environment information, it may be ascertained whether and/or how much material of at least one specified type of material is present in a cavity of the third article, and/or whether and/or in which surface portion of an outer surface of the third article it makes contact with an inner surface of a cavity of the patient that receives the third article. In addition or as an alternative, the item of environment information may relate to a surface structure of the inner surface of the cavity of the patient.

For example, as an item of environment information, it may be ascertained whether an object of the specified type of material (e.g., a calcification or a gallstone or a fragment thereof) has been received in the cavity of the third article or how large this object is. Identification of material that is to be removed in the cavity of the third article may be relevant, for example, when an endoscopy basket is used.

In addition or as an alternative, the item of environment information may relate to at least one of the following types of material: blood, blood clot, bone cement, an embolization material used to fill an aneurysm, and/or digestion fluid. For example, metal wire (e.g., a coil) or an initially liquid embolization material that hardens on contact with blood may be taken into consideration as the embolization material (e.g., an ethylene vinyl alcohol copolymer (EVOH), as is sold, for example, under the name Onyx®).

To identify contact of the outer surface of the third article with the inner surface of the cavity of the patient, it is possible to differentiate, for example, between different materials potentially arranged adjacent to the outer surface (e.g., between a wall material and blood or a digestion fluid), and/or forces that act on the outer surface may be identified and evaluated. Based on the surface structure of the cavity wall (e.g., a vessel wall), it is possible to identify, for example, a tumor and/or a calcification in the region of the cavity wall. The surface structure may be ascertained, for example, based on the shape of an elastically deformable third article, ascertained by the X-ray-dependent model, or the forces that act on the third article. However, in addition or as an alternative, for classification of the surface structure, it is also possible to take into consideration electrical items of information ascertained as an item of sensor information (e.g.,. measured potentials, an inductance, and/or a resistance).

The presence of a solid in a cavity of the third article and/or a control signal for an actuator in order to move the third article and/or a filling level of an aneurysm with an embolization material may be ascertained as an item of environment information or as an item of processing information ascertained based on the item of environment information.

For example, it may thus be ascertained whether a gallstone or a fragment thereof is present in an endoscopy basket that forms the third body. The presence of a solid may be identified, for example, based on the ascertained shape of the third article or based on ascertained forces on the third article, with it being possible to ascertain these variables, as explained above.

The control signal may specify, for example, a motor current for retrieving the third article (e.g., an endoscopy basket). For example, a force exerted by the third article on a solid received in the endoscopy basket may be ascertained in this connection as the item of environment information in order to regulate the exerted force via appropriate control of the motor current and thus prevent, for example, undesirable breakup of the solid or in order to purposefully cause such breaking up.

Such a force may be ascertained, for example, based on an ascertained deformation of the third article, and/or force sensors may directly measure at least a force on the third article. Based on the geometry of the action of the force, which is specified by the model of the third article, it is possible to infer the force acting locally on the solid from this.

The sensor or at least a respective one of the sensors, measured values of which the item of sensor information is based, may be a force sensor and/or a pressure sensor and/or a deformation sensor and/or an electrical sensor for capturing an electrical resistance and/or an electrical impedance and/or an electrical potential.

Using a force sensor and/or a pressure sensor and/or a deformation sensor, it is possible to extract, for example, additional items of information with respect to the shape of a deformable third article or the forces that generate it. For example, a potential underdetermination of the three-dimensional model resulting with exclusive evaluation of the X-ray image may be overcome hereby, and thus, the actual shape of the third article may be identified considerably more accurately. Based on this shape or the forces that act on the third article, pressures, etc., it is possible to quantify the mechanical interaction of the third article with material located in the surroundings of the third article (e.g., with material that is located in a cavity of the third article and/or with material that adjoins the outer wall thereof). The shape, for example, of a cavity of the body that receives the third article may be inferred hereby, whereby, for example, calcifications in a vessel may be identified. As has already been explained above, based on the shape or the forces, it is also possible to ascertain the presence of a solid in a cavity of the third article, a degree to which the third article touches a wall of the cavity of the patient, and/or other relevant items of environment information.

Based on known mechanical properties of the third article, it is also possible to infer properties of the material located in the surroundings of the third article, so it is possible to differentiate, for example, between liquid and solid materials in the environment of the third article (e.g., between vessel walls and blood that make contact with the outer wall of the third article), or even between different solid materials. Further, in the case of solid materials (e.g., in the case of vessel walls, calcifications, and blood clots), at least in the region in which the solid materials make contact with the third article, it is possible to infer their shape, so, for example, a deformation of a vessel wall due to a tumor or a calcification may be identified, and the size of an object received in an endoscopy basket may also be estimated and be provided as an item of environment information.

By capturing an electrical resistance or potential or an impedance of the material in the surroundings of the third article using the sensor, it is possible to differentiate, for example, different materials in order, for example, to differentiate whether exclusively fluid (e.g., blood or digestion fluid or bile) or a solid object (e.g., a gallstone or a calcification) has been received in an endoscopy basket. The actual position of the respective sensor or the sensor components of the sensor is accurately known based on the three-dimensional model parameterized by the X-ray image. This is relevant since electrical potentials may depend greatly on the specific measuring position and since with resistance measurements, the relative position of the points, between which the resistance is measured (e.g., its distance), is highly relevant. With induction measurements, it is expedient, for example, to take into consideration influencing of the inductance by the third article itself and thus by the shape thereof, and this is likewise possible based on the X-ray-dependent model.

In one embodiment, at least one fiber-optic shape sensor (e.g., based on a Faser-Bragg grating), or as an alternative or in addition, at least one strain gauge and/or at least one piezo-bending sensor, may be used as the deformation sensor.

In the X-ray image, a cavity that receives the third article of the patient may be segmented, and/or based on the X-ray image, a three-dimensional environment model of the environment of the third article may be parameterized as a function of the X-ray image. First, model parameters of the three-dimensional model of the third article are determined, or the possible parameter values of these model parameters are limited; and/or second, the item of environment information is additionally determined as a function of the segmentation of the cavity of the patient and/or the environment model.

The segmented cavity of the patient may be, for example, a vessel, a bile duct, or a section of the stomach or intestine. For example, the geometry of the segmented cavity of the patient may be taken into consideration in the framework of determining or limiting the model parameters. For example, parameterization of the model of the third article may additionally make use of the boundary condition that a forward projection of the third article is located inside the segmented cavity of the patient when the known imaging geometry is used. Unrealistic parameterizations of the three-dimensional model, in which the modeled third article would protrude beyond the cavity of the patient, may be ruled out hereby.

However, it is, for example, also possible to evaluate image contents that adjoin the segmented cavity of the patient. This may provide, for example, additional items of information for classification of material adjoining the third article, whereby, for example, calcifications and/or tumors adjacent to the cavity of the patient may be robustly identified.

Since, in general, extensive prior knowledge about the anatomy of patients and, further, typically further properties of the patient (e.g., height, weight, and gender), as well as three-dimensional preoperative image data such as CT or MR image datasets are known, a three-dimensional environment model with relevant items of information may already be generated based on a single X-ray image (e.g., via an elastic deformation of an anatomical atlas), with it being possible for particular features (e.g., tumors) to be identified and localized at least in the directions parallel to the image plane of the X-ray image.

A plurality of features of the third article may be specified, where the model positions of the features in the three-dimensional model depend on the model parameters. An image position in the X-ray image is allocated to the respective model position by a known mapping geometry of the respective X-ray image. Via a feature identification, at least one subgroup of the specified features is identified in the X-ray image, and its actual position in the X-ray image may be ascertained. The model parameters may be ascertained by optimizing a cost function that depends on a respective distance of the image position of the respective feature from the actual position of this feature.

The described procedure provides that the three-dimensional model may replicate the items of information, obtained via the X-ray image, with respect to the positions of the features or at least not deviate too greatly from them. As alternative or in addition, the cost function may depend on a quantity for a similarity of the mapping of the respective identified feature in the X-ray image to a mapping of the feature expected based on the three-dimensional model. For example, the shape of a feature in the three-dimensional model may be taken into consideration hereby.

As a function of the model parameters, the three-dimensional model may specify positions of a plurality of model points in the three-dimensional space, where the three-dimensional model or a specified calculation rule, as a function of the relative positions of at least one subgroup of the model points, specifies at least one item of model information. The model parameters may be ascertained by optimizing the or a cost function that depends on a quantity for the distance of the respective item of model information from the item of sensor information or a respective item of partial information of the item of sensor information. For example, at least some of the model points may be arranged at the model positions discussed above.

To take the item of sensor information into consideration when the X-ray-dependent model is parameterized, for example, the values of the model parameters may thus be varied to jointly minimize, by optimizing a joint cost function, the deviation of the model from the X-ray image, at least with respect to the positions of identified features, and the deviation of the item of model information, which would be expected in accordance with the resulting model for the items of sensor information, from the item of sensor information.

A correlation between model and item of sensor information may then be produced particularly easily when the item of sensor information or an item of partial information of the item of sensor information relates to a deformation of the third article, which may be captured, for example, by a bending sensor, or forces that act on the or inside the third article and may be captured, for example, by a force sensor. In general, the correlation between the relative position of various model points and the acting forces may be ascertained, for example, via the finite element method. However, a large number of third articles relevant in the context of medical imaging may also be sufficiently accurately described by simpler models (e.g., by a system of mass points coupled by springs and/or flexible bars). A description of this kind may be very suitable, for example, for describing a third article that is substantially composed of a wire mesh.

One example of a model for a complex article (e.g., a human body) is known, for example, from the publication Shetty, Karthik, et al., “BOSS: Bones, organs and skin shape model,” Computers in Biology and Medicine 165 (2023). The approaches explained there may also be used for the description of complex third articles parameterized by model parameters

The X-ray-dependent model and/or the model parameters and/or the three-dimensional environment model may be specified, and/or the item of environment information may be ascertained by a respective function trained by machine learning. In general, a function trained by machine learning mimics the cognitive functions that humans associate with thought processes of other humans. By way of training based on training data, the trained function is capable, for example, of adjusting to new circumstances and identifying and extrapolating patterns. Another possible designation for a “trained function” is a “model trained by machine learning.”

Although trained functions may learn complex correlations, the application of complex trained functions is also frequently at least approximately possible in real time. In the framework of training, the at least one trained function may thus learn, for example, to provide results that substantially match the results of a more computing—and/or memory-intensive ascertainment method used during training or also match results of an evaluation carried out or supported by an expert. Thus, by using a trained model, a result may be attained that is just as good as by way of manual analysis of the X-ray image and the item of sensor information by an expert or by a computing method that is more laborious in relation to the application of the trained function.

For example, the optimization, explained above, using a finite element method, that, depending on the desired model resolution, may be very computing-intensive and is thus unsuitable in some specific applications for real-time applications, may thus be used in the framework of training in order to provide X-ray-dependent models for training datasets. The trained function may learn to achieve similar results. This may make it possible to ascertain the item of environment information with a short delay of, for example, less than a second, so the ascertainment of the item of environment information may be used, for example, in the framework of fluoroscopy or for example to support an operation.

In general, the parameters of a machine learning model may be adjusted via training in order to provide the trained model. The training may, for example, precede the method of the present embodiments and are thus not part of the method of the present embodiments. For example, a method for training a trained function is thus also disclosed that is used to implement the specification of the X-ray-dependent model and/or the model parameters and/or the three-dimensional environment model and/or the ascertainment of the item of environment information in the method of the present embodiments. However, alternatively, it would also be possible to carry out the training as additional preceding method acts within the method of the present embodiments.

For example, supervised training, semi-supervised training, unsupervised training, reinforcement learning, and/or active learning may be used. Further, representation learning, which is also referred to as “feature learning,” may also be used. For example, the parameters of machine learning models may be iteratively adjusted by a plurality of training steps. For example, a particular cost function may be minimized in the framework of training. For example, for a neural network, the backpropagation algorithm may be used during training.

A machine learning model may include, for example, a neural network, a Support Vector Machine, a decision tree, and/or a Bayesian network, and/or a transformer, and/or the machine learning model may be based on k-means clustering, Q-learning, genetic algorithms, and/or association rules. A neural network may be, for example, a deep neural network, a convolutional neural network, or a convolutional deep neural network. In addition, a neural network may be an adversarial network, a deep adversarial network, and/or a generative adversarial network.

The third article may be an implant or a medical instrument. The item of environment information may thus serve, for example, to assess correct positioning of an implant after a procedure or a condition of an implant that has already been in the patient for some time and/or provide medical personal with information with respect to the environment of a medical instrument that may be used, for example, in the framework of a medical procedure (e.g., independently of the method of the present embodiments).

The item of environment information may, as already explained above in more detail, as a material, relate to a calcification and/or a gallstone and/or blood and/or a blood clot and/or bone cement and/or an embolization material that serves to fill an aneurysm, and/or digestion fluid and/or bile.

Apart from the method of the present embodiments, the present embodiments relate to a processing facility that is configured to carry out the computer-implemented method of the present embodiments. The processing facility may be configured, for example, as appropriately programmed data processing apparatuses, or the functionality may alternatively be implemented at least partly in a hard-wired manner. The processing facility may be integrated in a medical imaging facility (e.g., in an X-ray facility) or be configured separately from the medical imaging facility. The processing facility may be implemented, for example, as a workstation computer, server, or Cloud solution.

In addition, the present embodiments relate to an endoscopy facility with a third article configured for insertion into a patient, an actuator for moving the third article, and an inventive processing facility. The processing facility is also configured to control the actuator as a function of the item of environment information. The endoscopy facility may include, for example, an X-ray facility that may provide the X-ray image.

For example, the actuator may be a motor that serves to retrieve an endoscopy basket that forms the third article. As already explained in detail above, a motor current may, for example, be regulated such that a force on a solid (e.g., a gallstone) in the endoscopy basket is ascertained as an item of environment information and is regulated to a desired value by the motor current specification.

The present embodiments relate, for example, to a computer program with commands that are configured to carry out the computer-implemented method of the present embodiments when the commands are executed on a data processing apparatus.

Further, the present embodiments relate to a data carrier that includes the computer program of the present embodiments.

Feature and details of the methods of the present embodiments explained may also be transferred with the advantages to the further subject matters of the present embodiments, and vice versa.

Independent of the grammatical term usage, individuals with male, female, or other gender identities are included within the term.

### BRIEF DESCRIPTION OF THE DRAWINGS

Further advantages and details of the invention are found in the following example embodiments as well as the associated drawings. In the drawings, schematically:

FIG. 1 shows an example embodiment of an endoscopy facility that includes an example embodiment of a processing facility;

FIG. 2 shows a flowchart of an example embodiment of a method;

FIGS. 3-5 show example X-ray images in illustrative usage situations of embodiments of the method; and

FIG. 6 shows an illustrative structure of a function trained by machine learning.

### DETAILED DESCRIPTION

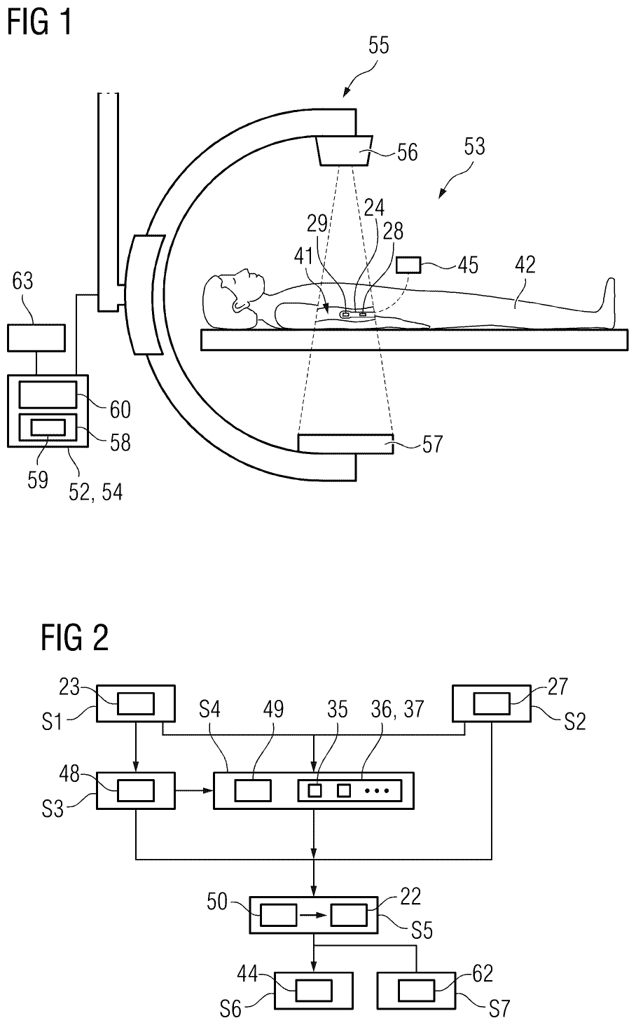

FIG. 1 schematically shows an endoscopy facility 53 with a third article 24 embodied for insertion in a patient 42, an actuator 45 for moving the third article 24, and a processing facility 52 (e.g., including one or more processors). The endoscopy facility 53 includes an X-ray facility 55 in order to capture, using an X-ray source 56 and an X-ray detector 57 (e.g., in the framework of fluoroscopy), at least one X-ray image 23 that, in the usage situation shown, maps the third article 24 located inside a patient 42.

As will be explained in more detail later based on the illustrative embodiment of this type of processing shown in FIG. 2, the X-ray image 23 together with an item of sensor information 27, which is based on at least one respective measured value of the sensors 28, 29 of the third article 24, is processed by the processing facility 52 in order to provide an item of environment information 22 that relates to a material 32 in surroundings of the third article 24 or an interaction of the third article 24 with the material 32.

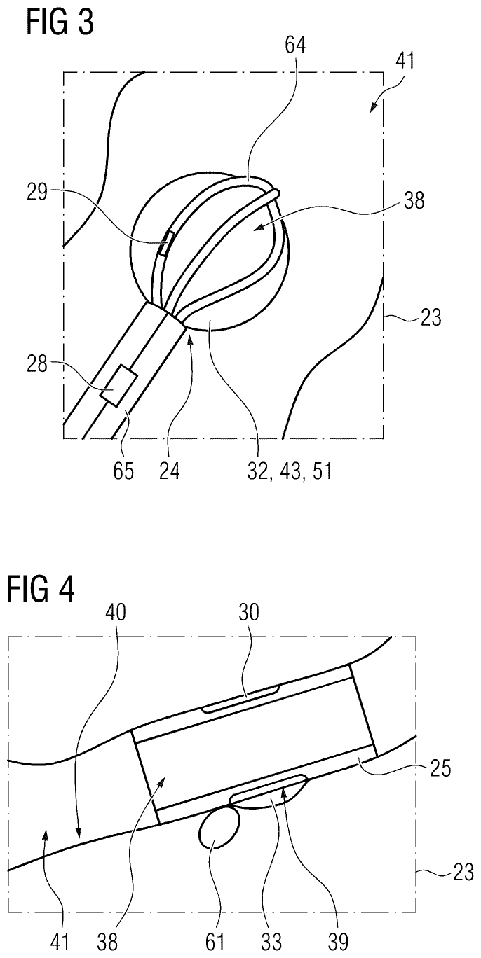

As will be explained later based on the individual illustrative usage situations shown in FIGS. 3-5, the item of environment information 22 may be used, for example, to output to a user (e.g., via the display facility 63) an overlaid representation 62 of the X-ray image 23 with the item of environment information 22 or an item of information derived herefrom. For example, a specific material or an identified article made from the material may be highlighted in color there. In addition or as an alternative, the processing facility 52 may actuate the actuator 45 as a function of the item of environment information 22. This may serve, for example, to optimally position a third article 24-26 (e.g., a stent) or also to control retrieval of a third article 24-26 (e.g., an endoscopy basket).

The method is implemented in the example by a computer program 59 that is stored in a memory 58 of a data processing apparatus 54. Execution of the commands of the computer program 59 by the processor 60 of the data processing apparatus 54 carries out the method, or this programming of the data processing apparatus 54 implements the processing facility 52.

An illustrative embodiment of a method for ascertaining the item of environment information 22 will be explained below with reference to the flowchart shown in FIG. 2. In this context, the illustrative application situations of this method, represented in FIGS. 3-5 as a respective schematic X-ray image 23, will also be discussed. The three application situations shown are purely illustrative, and the method may basically be used for a large number of further specific applications, as already discussed in the general part of the description.

In the example, first, at least one X-ray image 23 is captured in act S1, and this maps at least a portion of a third article 24-26 located inside a patient 42. In the example of FIG. 3, an endoscopy basket is mapped as the third article 24; in the example of FIG. 4, a stent is mapped as the third article 25; and in the example of FIG. 5, an endoscope 26 for introducing an embolization material 47 (e.g., a platinum coil) is mapped as the third article 26. The further steps will first be explained with a focus on the example of FIG. 3, according to which some deviations and additions for the further examples of FIGS. 4 and 5 will be discussed.

In the example shown in FIG. 3, it should first be identified whether a solid 43 that is to be removed from the body (e.g., a gallstone 51) has been successfully received by the endoscopy basket 24. Second, if this is the case, the retractor of the third article 24 may be controlled by the actuator 45 via an appropriate rotor current specification.

In act S2, an item of sensor information 27 is ascertained for this purpose, which, in the case shown in FIG. 3, is based on at least one respective measured value of the sensors 28, 29 of the third article 24. In the example, the sensor 29 is an inductance sensor, and the sensor 28 is a force sensor integrated in a control wire.

In act S3, a three-dimensional environment model 48 of the respective environment of the respective third article 24-26 is then parameterized based on the X-ray image 23 as a function of the X-ray image 23, which describes at least the cavity 41 of the patient 42 that receives the third article 24-26. This may take place, for example, in that the X-ray image is elastically registered with an anatomical atlas or with three-dimensional image data captured in advance. Known properties of mapped biological material (e.g., a known elasticity of tissue) may also be taken into consideration in this connection. Similar to the ascertainment of the X-ray-dependent model of the third article explained below, the model parameters may also be determined by a trained function that is trained, for example, with the aid of training datasets that are based on three-dimensional imaging procedures.

To reduce the complexity of the method, it would, for example, also be possible in act S3 to segment solely the cavity 41 of the patient 42 that receives the third article 24-26. In principle, consideration of the environment represented in the X-ray image 23 may also be omitted completely.

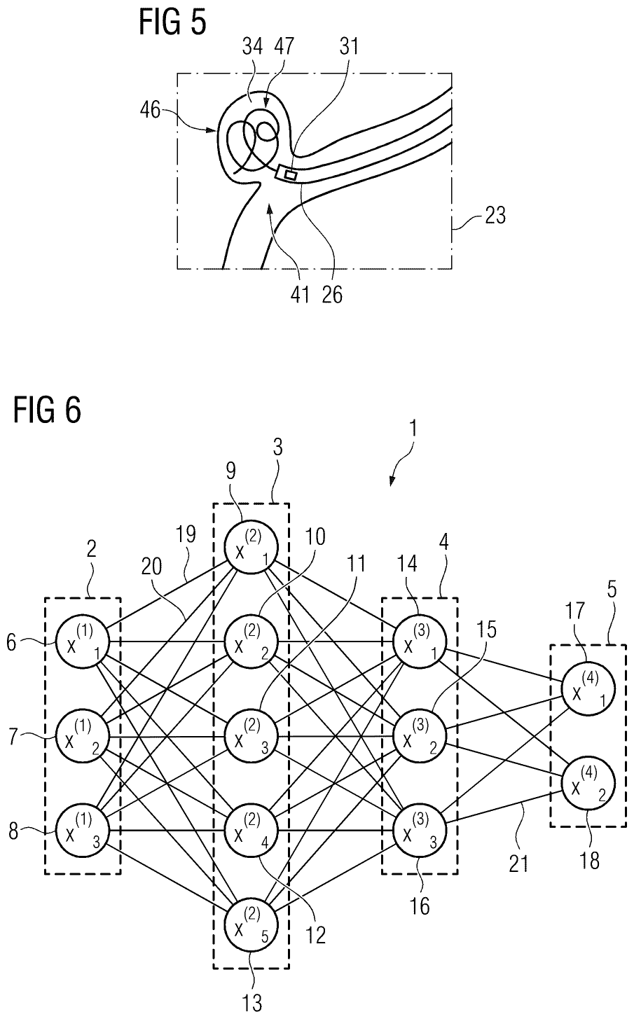

In act S4, model parameters 35 of a three-dimensional model 36 describing the third article 24 are subsequently determined as a function of the X-ray image 23 and, in the example, additionally as a function of the item of sensor information 27 and of the environment model 48, in order to provide an X-ray-dependent model 37. Although a trained function 49 is used for this purpose in the example shown, for which function one possible implementation will be explained below with reference to FIG. 6, for a better understanding of the correlations used or identified here, first, a different, although typically more computing-intensive approach, will be explained below for ascertaining these model parameters 35.

In order to ascertain the model parameters, it is possible to specify a specified list of features of the third article 24 that may potentially be identified in the X-ray image. One model position is allocated to each feature of the specified list of features in the three-dimensional model. The model positions depend on the model parameters. Suitable features are, for example, the ends of the wires 64 of the endoscopy basket, points of maximum and minimum curvature, and points that have the maximum spacing from a center line of a guide 65. An image position in the X-ray image 23 is allocated to the respective model position by a known mapping geometry of the respective X-ray image 23.

At least some of these features may be identified in the X-ray image 23, and their actual positions in the X-ray image 23 may be ascertained via a feature identification of at least one subgroup of the specified features in the X-ray image 23 (e.g., via edge detection) by using scale-invariant features or via a trained algorithm. The model parameters may subsequently be ascertained by minimizing a cost function that depends on a respective spacing of the image position of the respective feature from the actual position of this feature.

As has already been explained in the general part, a respective quantity for the similarity of shapes of parts of the third article 24 found in the X-ray image (e.g., of the wires 64) to the shape that results with a forward projection of the corresponding part of the model in the image plane of the X-ray image may also be taken into consideration in the cost function.

For example, by using the finite element method as the calculation rule, a correlation between the force that acts on the endoscopy basket and is captured by the sensor 28, and the resulting shape of the endoscopy basket may also be known. Therefore, as a function of the model parameters 35, an item of model information that corresponds to a value of the item of sensor information 27 or the item of partial information of the item of sensor information 27 relating to the sensor 28 that is expected with given values of the model parameters 35 may be ascertained. By using a cost function that also depends on a quantity for the spacing of the respective item of model information from the item of sensor information 27 or the partial information, it is thus possible to achieve a more accurate parameterization of the three-dimensional model, or ambiguities, which may result in the case of sole consideration of the X-ray image 23, may be removed.

In addition, the inductance ascertained by the sensor 29 may also be taken into consideration when ascertaining the model parameters 35. Based on an inductance ascertained as part of the item of sensor information 27, it is, for example, possible to identify whether a solid 43 (e.g., a gallstone 51) is located inside the cavity 38 of the endoscopy basket, due to the presence of which additional forces act on the third article 24.

Based on the three-dimensional environment model 48, an additional boundary condition is specified in the example for the X-ray-dependent model 37 of the third article (e.g., that the third article 24-26 is located inside the cavity 41 of the patient 42). A similar boundary condition may also be obtained if a two-dimensional segmentation of the cavity 41 has taken place in act S3. In this case, it may be stipulated, for example, that a forward projection of the X-ray-dependent model 37 of the third article is located inside the segmented cavity 41 according to the known imaging geometry of the X-ray image 23.

Since the described procedure may be connected with high computing effort in the framework of an optimization (e.g., owing to the use of the finite element method), a simpler model of the third article 24 may be used instead, as has already been explained in the general part, or, as schematically represented in FIG. 2, a trained function 49 may be used for ascertaining the model parameters 35 instead when using the method in the field. A neural network, by way of example, is used as the trained function 49, where the principle underlying a neural network will be explained in more detail later with reference to FIG. 6. For example, the X-ray image may first be processed by convolutional layers, with it being possible for the result of this pre-processing, together with the item of sensor information, to be supplied to one or more fully connected layers.

In the example, the trained function 49 is based on supervised learning with backpropagation, which is also referred to as a backpropagation algorithm, using the gradient descent method. Training datasets used for this may include the respective X-ray image and the items of sensor information as input data and the model parameters as the desired output data. In principle, the model parameters may be indicated by a medical specialist who analyzes the input data, with it being possible to take into consideration, for example, items of additional information (e.g., items of information of a three-dimensional imaging of the third article 24-26). However, it is also possible that at least some of the desired output data is ascertained from the input data using the robust, but relatively more computing-intensive, approach explained above, so the trained function 49 learns to provide similarly good results to the procedure explained above with potentially noticeably lower computing effort.

In act S5, the item of environment information 22 is ascertained as a function of the X-ray-dependent model 37 and the item of sensor information 27. In the example shown in FIG. 3, the ascertained item of environment information may, in the simplest case, describe whether a specific solid 43 (e.g., a gallstone 51) is present in the cavity 38 of the third article 24 (e.g., inside the endoscopy basket). Since the instantaneous shape of the endoscopy basket is already known through the X-ray-dependent model 37, it is sufficient for this purpose to differentiate between a filling of the cavity with digestion fluid or bile and an at least partial displacement of the digestion fluid or bile from the cavity 38 by the gallstone 51. Since these states result in inductances that differ noticeably from one another, a gallstone 51 may be identified in the endoscopy basket, for example, as a function of whether the inductance captured by the sensor 29 overshoots a limit value specified as a function of the X-ray-dependent model 37.

Since the volume and the shape of the cavity 38 of the third article 24 are specified by the X-ray-dependent model 37, a volume of the gallstone 51 may also be estimated, or be generally estimated, based on the captured inductance, how much material 32-34 of at least one specified type of material (e.g., gallstone material or digestion fluid) is present in the cavity 38 of the third article 24-26. In addition, the environment model 48 or also a segmentation of the gallstone 51 may optionally be used in the X-ray image 23 to estimate its dimensions in the image plane and its position.

Based on these items of information and the ascertained shape of the third article 24 and the force ascertained by the sensor 28, it is also possible to estimate the force that is currently acting on the gallstone 51.

As represented above, for example, the force that is acting on the gallstone, ascertained in the example, and its ascertained size depend on a large number of measured variables and model parameters 35. In principle, the correlations of the various input variables may be ascertained with the respective output variable via experiments (e.g., by preliminary tests carried out outside of the body, in which, for example, three-dimensional X-ray data may also be used as a supplementary information source). The results of these preliminary tests may be analyzed, for example, by a regression analysis in order to provide an analytical correlation for calculation of the respective output variable from the input variables. However, appropriate correlations may frequently be ascertained with a lower number of input data or preliminary tests if machine learning is used. Therefore, a trained function 50 is used in the example to ascertain the item of environment information 22.

The trained function 50, just like the trained function 49 explained above, may be implemented, for example, by a neural network that is trained by supervised training using training datasets (e.g., using a gradient descent method). For example, the preliminary tests explained above may be carried out to provide training datasets.

The ascertained item of environment information 22 is used in act S6 in the example of FIG. 3 to specify a control signal 44 for the actuator 45 schematically represented in FIG. 1 in order to move the third article 24 (e.g., to retrieve the endoscopy basket after receiving the gallstone 51). In the example, the control signal 44 specifies a motor current of the actuator 45. The motor current is set such that the force ascertained as an item of environment information 22, that acts on the gallstone 51, is regulated to a desired value to first make swift retrieval of the endoscopy basket possible, and second to provide that it is not broken by an excessive action of the force. If acts S1-S6 are repeatedly carried out (e.g., in the framework of fluoroscopy that supports an operation), the motor current may thus be almost continuously regulated hereby.

In act S7, an overlaid representation 62 of the X-ray image 23 and the dimensions of the gallstone 51 ascertained as an item of environment information 22 is generated, which may be output to a user via the display facility 63. It is also possible that in a modification of the method, solely, the actuator 45 is controlled according to act S6, or the information is output to a user according to act S7 as a function of the environment situation 20.

The basic procedure, explained with reference to FIG. 2, may also be used in a large number of other application situations in order to ascertain a respective item of environment information 22 with respect to the environment of a respective third article 24-26. X-ray images 23 for further application situations are schematically represented in FIGS. 4 and 5.

In FIG. 4, as an item of environment information 22 for a stent as the third article 25, it is ascertained in which surface portion of an outer surface 39 of the stent the stent makes contact with an inner surface 40 of the cavity 41 of the patient 42 that receives the third article 35 (e.g., a blood vessel). In the example shown, based on the X-ray image 23, it is already possible to identify that in a portion of the stent, a free space remains 33 between the stent and the inner surface 40. However, based on the X-ray image or a parameterization of the environment model 48 based solely on the X-ray image, or the X-ray-dependent model 37 of the third article 25, it would not be possible to clearly identify to what extent this free space 33 extends in the circumferential direction of the stent.

Therefore, items of sensor information of the sensor 30 are additionally evaluated. The sensor 30 may be an impedance sensor. Since blood and tissue in the surroundings of the sensor 30 result in the significantly different captured impedance, items of information about the extent of the free space 33 outside of the image plane may also be extracted by the joint use of the X-ray-dependent model 37 and the item of sensor information 27 that is provided here.

Alternatively, it would also be possible, for example, to use a force sensor as the sensor 30 in FIG. 4, via which forces that act, for example, on different segments in the circumferential direction of the stent are captured. It is hereby possible to differentiate whether a respective circumferential segment of the stent abuts the vessel wall or adjoins the free space 39.

The identification of forces also makes it possible to ascertain, for example, a surface structure of the inner surface 40 of the cavity 41 of the patient 42 as an item of environment information 22. For example, the tumor 61 schematically represented in FIG. 4 may have a different tissue elasticity than the surrounding tissue, so in that portion of the third article 25 that adjoins the tumor 61, forces different from other regions act on the outer surface 39 of the third article 25.

In the example shown in FIG. 5, an endoscope for introducing an embolization material 47 (e.g., a platinum coil) is mapped as the third article 26. Based on the X-ray image 23 or the environment model 48 ascertained herefrom, and X-ray-dependent model 37 of the third article 26, it is possible to identify that the endoscope is arranged in the region of the aneurysm 46 and that embolization material 47 has already been introduced into the region of the aneurysm. However, owing to the scatter and absorption behavior of the metal used as embolization material 47, based on the X-ray image, it is frequently not possible to identify whether an adequate filling level of the aneurysm 46 has already been achieved. In addition to the X-ray-dependent model 37 of the third article 26, at least one measured value of the sensor 31, which is an impedance sensor in the example, may thus be used as an item of sensor information in order to ascertain the filling level of the aneurysm 46 as an item of environment information 22.

Since the inductance measured by the sensor 31 depends to a large extent on the distance of the sensor 31 from the aneurysm and the accumulation of embolization material 47 received therein, for ascertaining a robust item of environment information 22, it is also necessary to take the position of the sensor 31 known from the X-ray-dependent model 37 into consideration.

As has already been explained above, the trained functions 49, 50 in the example shown in FIG. 2 may be implemented by neural networks. For the sake of simplicity, the properties of such a neural network will be briefly explained below with reference to FIG. 6 based on a very simple example, with it being possible to use considerably larger numbers of input nodes, output nodes, and layers in real implementations. English expressions for the artificial neural network 1 are “artificial neural network,” “neural network,” “artificial neural net,” or “neural net.”

The artificial neural network 1 includes nodes 6 to 18 and edges 19 to 21, with each edge 19 to 21 being a directed connection from a first node 6 to 18 to a second node 6 to 18. In general, the first node 6 to 18 and the second node 6 to 18 are different nodes 6 to 18; however, it is also possible that the first node 6 to 18 and the second node 6 to 18 are identical. For example, in FIG. 1, the edge 19 is a directed connection from the node 6 to the node 9, and the edge 21 is a directed connection from the node 16 to the node 18. An edge 19 to 21 from a first node 6 to 18 to a second node 6 to 18 is referred to as an ingoing edge for the second node 6 to 18 and as an outgoing edge for the first node 6 to 18.

In this example embodiment, the nodes 6 to 18 of the artificial neural network 1 may be arranged in layers 2 to 5, with it being possible for the layers to have an intrinsic order that is introduced by the edges 19 to 21 between the nodes 6 to 18. For example, edges 19 to 21 may be provided only between adjacent layers of nodes 6 to 18. In the represented example embodiment, there exists an input layer 2 that has solely the nodes 6, 7, 8, in each case without ingoing edge. The output layer 5 includes only the nodes 17, 18, in each case without outgoing edges, with hidden layers 3 and 4 being located between the input layer 2 and the output layer 5. In the general case, the number of hidden layers 3, 4 may be arbitrarily selected. The number of nodes 6, 7, 8 of the input layer 2 may correspond to the number of input values in the neural network 1, and the number of nodes 17, 18 in the output layer 5 may correspond to the number of output values of the neural network 1.

For example, a (real) number may be allocated to the nodes 6 to 18 of the neural network 1. In this case, x⁽ⁿ⁾i denotes the value of the i^{th} node 6 to 18 of the n^{th} layer 2 to 5. The values of the nodes 6, 7, 8 of the input layer 2 are equivalent to the input values of the neural network 1, while the values of the nodes 17, 18 of the output layer 5 are equivalent to the output values of the neural network 1. Further, a weight in the form of a real number may be allocated to each edge 19, 20, 21. For example, the weight is a real number in the interval [−1, 1] or in the interval [0, 1,]. In this case, w^{(m, n)}_{i,j} denotes the weight of the edge between the i^{th} node 6 to 18 of the m^{th} layer 2 to 5 and the j^{th} node 6 to 18 of the n^{th} layer 2 to 5. Further, the abbreviation

$${w}_{i , j}^{( n )}$$

is defined for the weight

$${w}_{i , j}^{( n , n + 1 )} .$$

To calculate output values of the neural network 1, the input values are propagated by the neural network 1. For example, the values of the nodes 6 to 18 of the (n+1)^{th} layer 2 to 5 based on the values of the nodes 6 to 18 of the n^{th} layer 2 to 5 may be calculated by

$${x}_{j}^{( n + 1 )} = f ( {\sum }_{i} {x}_{i}^{( n )} \cdot {w}_{i , j}^{( n )} ) .$$

In this case, f is a transfer function that may also be referred to as an activation function. Known transfer functions are step functions, sigmoid functions (e.g., the logistical function, the generalized logistical function, the hyperbolic tangent, the arc tangent, the error function, the smoothstep function) or rectifier functions (e.g., rectifier). The transfer function is substantially used for standardization purposes.

For example, the values are propagated layer by layer by the neural network 1, with values of the input layer 2 being given by the input data of the neural network 1. Values of the first hidden layer 3 may be calculated based on the values of the input layer 2 of the neural network 1, values of the second hidden layer 4 may be calculated based on the values in the first hidden layer 3, etc.

To be able to define the values

$${w}_{i , j}^{( n )}$$

for the edges 19 to 21, the neural network 1 has to be trained using training data. For example, training data includes training input data and training output data that are referred to as tᵢ below. For a training step, the neural network 1 is applied to the training input data to ascertain calculated output data. For example, the training output data and the calculated output data include a number of values, with the number being determined as the number of nodes 17, 18 of the output layer 5.

For example, a comparison between the calculated output data and the training output data is used to recursively adjust the weights inside the neural network 1 (e.g., back propagation algorithm). For example, the weights may be changed in accordance with

$${w}_{i , j}^{′ ( n )} = {w}_{i , j}^{( n )} - γ \cdot {δ}_{j}^{( n )} \cdot {x}_{i}^{( n )}$$

where γ is a learning rate, and the numbers

$${δ}_{j}^{( n )}$$

may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on

$${δ}_{j}^{( n + 1 )}$$

if the (n+1)^{th} layer is not the output layer 5, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)^{th} layer is the output layer 5, where f is the first derivation of the activation function and

$${y}_{j}^{( n + 1 )}$$

is the comparison training value for the j^{th} node 17, 18 of the output layer 5.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for ascertaining an item of environment information based on at least one two-dimensional X-ray image that maps at least one portion of a third article located inside a patient, and at least one item of sensor information that is based on at least one measured value of at least one sensor of the third article, wherein the item of environment information relates to material in surroundings of the third article, an interaction of the third article with the material, or a combination thereof, the computer-implemented method comprising:
receiving the at least one two-dimensional X-ray image and the at least one item of sensor information;
determining model parameters or limiting possible parameter values of the model parameters of a three-dimensional model of the third article as a function of the at least one two-dimensional X-ray image in order to specify an X-ray-dependent model, wherein the three-dimensional model describes a three-dimensional shape, pose, or three-dimensional shape and pose of the third article as a function of the model parameters;
ascertaining the item of environment information as a function of the X-ray-dependent model, wherein the item of environment information, the X-ray-dependent model, or the item of environment information and the X-ray-dependent model also depend on the at least one item of sensor information; and
providing the item of environment information.

2. The computer-implemented method of claim 1, wherein the third article is deformable, and
wherein at least one of the model parameters describes the deformation.

3. The computer-implemented method of claim 1, wherein:
as the item of environment information, it is ascertained whether, how much, or whether and how much material of at least one specified type of material is present in a cavity of the third article, whether, in which surface portion of, or whether and in which surface portion of an outer surface of the third article makes contact with an inner surface of a cavity that receives the third article of the patient, or a combination thereof;
the item of environment information relates to a surface structure of the inner surface of the cavity of the patient; or
a combination thereof.

4. The computer-implemented method of claim 1, wherein as the item of environment information or as an item of processing information ascertained based on the item of environment information, presence of a solid in a cavity of the third article, a control signal for an actuator in order to move the third article, a filling level of an aneurysm with an embolization material, or any combination thereof is ascertained.

5. The computer-implemented method of claim 1, wherein the at least one sensor or a respective one of the at least one sensor, on which measured values the item of sensor information is based, is a force sensor, a pressure sensor, a deformation sensor, an electrical sensor, or any combination thereof for capturing an electrical resistance, an electrical impedance, an electrical potential, or any combination thereof.

6. The computer-implemented method of claim 1, wherein:
in the at least one X-ray image, a cavity that receives the third article of the patient is segmented;
based on the at least one X-ray image, a three-dimensional environment model of the environment of the third article is parameterized as a function of the at least one X-ray image; or
a combination thereof, and
wherein:
the model parameters of the three-dimensional model of the third article are determined, or the possible parameter values of the model parameters are limited;
the item of environment information is additionally determined as a function of the segmentation of the cavity of the patient, the environment model, or a combination thereof; or
a combination thereof.

7. The computer-implemented method of claim 1, wherein a plurality of features of the third article are specified,
wherein model positions of features in the three-dimensional model depend on the model parameters,
wherein an image position in the at least one two-dimensional X-ray image is allocated to the respective model position via a known mapping geometry of the respective X-ray image,
wherein via a feature identification, at least one subgroup of the specified features is identified in the at least one two-dimensional X-ray image, and an actual position of the at least one subgroup of the specified features is specified in the at least one two-dimensional X-ray image, and
wherein the model parameters are ascertained by optimizing a cost function that depends on a respective distance of the image position of the respective feature from the actual position of the respective feature.

8. The computer-implemented method of claim 1, wherein the three-dimensional model, as a function of the model parameters, specifies positions of a plurality of model points in the three-dimensional space,
wherein the three-dimensional model or a specified calculation rule, as a function of the relative positions of at least one subgroup of the model points, specifies at least one item of model information, and
wherein the model parameters are ascertained by optimizing a cost function that depends on a quantity for a distance of the respective item of model information from the item of sensor information or a respective item of partial information of the item of sensor information.

9. The computer-implemented method of claim 1, wherein the X-ray-dependent model, the model parameters, the three-dimensional environment model, or any combination thereof is specified, the item of environment information is ascertained by a respective function trained by machine learning, or a combination thereof.

10. The computer-implemented method of claim 1, wherein the third article is an implant or a medical instrument.

11. The computer-implemented method of claim 1, wherein the item of environment information as a material relates to a calcification, a gallstone, blood, a blood clot, bone cement, an embolization material, or any combination thereof serving to fill an aneurysm, digestion fluid, bile, or any combination thereof.

12. A processing facility comprising:
a processor configured to ascertain an item of environment information based on at least one two-dimensional X-ray image that maps at least one portion of a third article located inside a patient, and at least one item of sensor information that is based on at least one measured value of at least one sensor of the third article, wherein the item of environment information relates to material in surroundings of the third article, an interaction of the third article with the material, or a combination thereof, the processor being configured to ascertain the item of environment information comprising the processor being configured to:
receive the at least one two-dimensional X-ray image and the at least one item of sensor information;
determine model parameters or limit possible parameter values of the model parameters of a three-dimensional model of the third article as a function of the at least one two-dimensional X-ray image in order to specify an X-ray-dependent model, wherein the three-dimensional model describes a three-dimensional shape, pose, or three-dimensional shape and pose of the third article as a function of the model parameters;
ascertain the item of environment information as a function of the X-ray-dependent model, wherein the item of environment information, the X-ray-dependent model, or the item of environment information and the X-ray-dependent model also depend on the at least one item of sensor information; and
provide the item of environment information.

13. An endoscopy facility comprising:
a third article configured for insertion into a patient;
an actuator configured to move the third article; and
a processing facility comprising:
a processor configured to ascertain an item of environment information based on at least one two-dimensional X-ray image that maps at least one portion of a third article located inside the patient, and at least one item of sensor information that is based on at least one measured value of at least one sensor of the third article, wherein the item of environment information relates to material in surroundings of the third article, an interaction of the third article with the material, or a combination thereof, the processor being configured to ascertain the item of environment information comprising the processor being configured to:
receive the at least one two-dimensional X-ray image and the at least one item of sensor information;
determine model parameters or limit possible parameter values of the model parameters of a three-dimensional model of the third article as a function of the at least one two-dimensional X-ray image in order to specify an X-ray-dependent model, wherein the three-dimensional model describes a three-dimensional shape, pose, or three-dimensional shape and pose of the third article as a function of the model parameters;
ascertain the item of environment information as a function of the X-ray-dependent model, wherein the item of environment information, the X-ray-dependent model, or the item of environment information and the X-ray-dependent model also depend on the at least one item of sensor information; and
provide the item of environment information, and
wherein the processing facility is further configured to control the actuator as a function of the item of environment information.

14. In a non-transitory computer-readable storage medium that stores instructions executable by one or more processors to ascertain an item of environment information based on at least one two-dimensional X-ray image that maps at least one portion of a third article located inside a patient, and at least one item of sensor information that is based on at least one measured value of at least one sensor of the third article, wherein the item of environment information relates to material in surroundings of the third article, an interaction of the third article with the material, or a combination thereof, the instructions comprising:
receiving the at least one two-dimensional X-ray image and the at least one item of sensor information;
determining model parameters or limiting possible parameter values of the model parameters of a three-dimensional model of the third article as a function of the at least one two-dimensional X-ray image in order to specify an X-ray-dependent model, wherein the three-dimensional model describes a three-dimensional shape, pose, or three-dimensional shape and pose of the third article as a function of the model parameters;
ascertaining the item of environment information as a function of the X-ray-dependent model, wherein the item of environment information, the X-ray-dependent model, or the item of environment information and the X-ray-dependent model also depend on the at least one item of sensor information; and
providing the item of environment information.

15. The non-transitory computer-readable storage medium of claim 14, wherein the third article is deformable, and
wherein at least one of the model parameters describes the deformation.

16. The non-transitory computer-readable storage medium of claim 14, wherein:
as the item of environment information, it is ascertained whether, how much, or whether and how much material of at least one specified type of material is present in a cavity of the third article, whether, in which surface portion of, or whether and in which surface portion of an outer surface of the third article makes contact with an inner surface of a cavity that receives the third article of the patient, or a combination thereof;
the item of environment information relates to a surface structure of the inner surface of the cavity of the patient; or
a combination thereof.

17. The non-transitory computer-readable storage medium of claim 14, wherein as the item of environment information or as an item of processing information ascertained based on the item of environment information, presence of a solid in a cavity of the third article, a control signal for an actuator in order to move the third article, a filling level of an aneurysm with an embolization material, or any combination thereof is ascertained.

18. The non-transitory computer-readable storage medium of claim 14, wherein the at least one sensor or a respective one of the at least one sensor, on which measured values the item of sensor information is based, is a force sensor, a pressure sensor, a deformation sensor, an electrical sensor, or any combination thereof for capturing an electrical resistance, an electrical impedance, an electrical potential, or any combination thereof.

19. The non-transitory computer-readable storage medium of claim 14, wherein:
in the at least one X-ray image, a cavity that receives the third article of the patient is segmented;
based on the at least one X-ray image, a three-dimensional environment model of the environment of the third article is parameterized as a function of the at least one X-ray image; or
a combination thereof, and
wherein:
the model parameters of the three-dimensional model of the third article are determined, or the possible parameter values of the model parameters are limited;
the item of environment information is additionally determined as a function of the segmentation of the cavity of the patient, the environment model, or a combination thereof; or
a combination thereof.
